(19) United States
(12) Patent Application Publication
(10) Pub. No.: US 2021/0103939 A1
(43) Pub. Date: Apr. 8, 2021

(54) COMPUTERISED SYSTEM AND METHOD FOR MATCHING A USER TO A CAREGIVER OR A CAREGIVING FACILITY

(71) Applicants: Teresa Sullivan McLean, Guilford, CT (US); Brenda Limone, Topsfield, MA (US)

(72) Inventors: Teresa Sullivan McLean, Guilford, CT (US); Brenda Limone, Topsfield, MA (US)

(73) Assignee: LifestyleCX, Inc., Guilford, CT (US)

(21) Appl. No.: 16/595,993

(22) Filed: Oct. 8, 2019

(51) Int. Cl. G06Q 30/02; G16H 10/60; G06Q 40/08; G06Q 50/18; G06N 20/00; H04L 29/06

(52) U.S. Cl. CPC G06Q 30/0201; G16H 10/60; G06Q 30/0283; H04L 63/062; G06Q 50/18; G06N 20/00; G06Q 40/08

(57) ABSTRACT

A full end-to-end computerized method and system for matching a caregiver or a care giving facility to a patient is provided. In performing a match, the Care Matching System uses, among other things, input from the patient, such as patient needs, requirements, and preferences, sophisticated decision trees, real-time data from caregivers and caregiving facilitates, crowd sourced data, scoring mechanisms, and machine learning. In addition to matching a caregiver or a care giving facility to a patient, the Care Matching System provides features that manage patient onboarding, medical insurance verification, paperwork completion and submission. Further the Care Matching System provides tools for managing continued care through use of a decision matrix and deep learning.

In operation, the Care Matching System communicates with a patient's electronic device. Using this device, which may be a mobile phone or a laptop, the patient enters patient care information that includes patient location, housing options, personal care options, insurance options. The patient care information may also include patient's medical needs, patient's preferences, patient's requirements, patient's care options, patient's primary needs, patient's top concerns, patient's lifestyle interests, patient's ethnic and cultural requirements, patient's memory care, patient's nursing and rehabilitation care, patient's budget, patient's language preferences, and patient's care giver preference.

The Care Matching System establishes a connection between the electronic device and a server of a Care Matching System over a network. The server of the Care Matching System includes a processor and memory for storing patient care data.

As an initial step, the Care Matching System receives patient care information entered by the patient. The patient could enter this information through a downloadable mobile application or connect online to a wizard that provides selectable options.

The processor compartmentalizing the patient care information into a plurality of categories. For example, these categories could be housing, personal care, insurance, memory care or other options mentioned above.

The Care Matching System also establishes a connection over a network with a plurality of client computing devices connected to either a care giver or a care giving facility.

The processor performs a search query of care givers or care giving facilities within a predetermined distance from the patient location and receives responses to the query them.

The processor analyses each care giver or care giving facility for its response relating to each category and scores the response. The scoring is weighted between a low, medium, or high with low receiving a lower weight than a high. Alternatively, the processor analyses the response and determines a percentage match between the desired requirements entered by the patient through their electronic device and the offering of the care giving facility.

All the scores for each category are calculated to produce a final score for each care giver or care giving facility. Upon final scoring, the care givers and care giving facilities are ranked and displayed on the patient's electronic device.

The patient can review the different types of care recommended as well as the recommended ranked care givers and care giving facilities and select one of the care givers or care giving facilities for onboarding. The onboarding process provides sign off on all required paperwork for admission and also schedules an admission date.

The end-to-end computerized method also uses crowd sourcing and deep learning to refine and enhance the listing of matched care givers or care giving facilities.

When using crowd sourcing, the method includes the steps of receiving patient care information entered into the electronic device by the patient that needs the care. Then it selects one category of patient care information from the patient care information. It also establishes a connection with a plurality of patients within a vicinity from the location of the electronic device and queries them to respond in relation to a category. The plurality of patient provides insight and customer satisfaction data for their care and for the care giving facility they used. As a next step, category related information is extracted from the response and a score for the care giver or a caregiving facility.

Similarly, the method also uses deep learning. In operation, the deep learning engine establishing a connection with the crowd sourcing engine and obtains data from the crowd sourcing engine on each query by the plurality of patients and their responses by the car givers or the care giving facilities. The deep learning engine then refines the ranked care givers and care giving facilities by either upgrading their rank.

As a result of the queries, scoring, analysis, crowd sourcing, and deep learning, a patient is matched with a care giver or care giving facility that need their medical needs, requirements, preferences to hopefully increase their customer satisfaction.

### COPYRIGHT NOTICE

A portion of the disclosure of this patent document contains material that may be subject to copyright protection. The copyright owner has no objection to the facsimile reproduction by anyone of the patent document or the patent disclosure as it appears in the Patent and Trademark Office patent file or records, but otherwise reserves all copyright rights.

### TECHNICAL FIELD

The present application relates to computing systems that use decision matrix, crowd sourcing, and deep learning to analyze data and match a user (or patient) to a caregiver or a caregiving facility.

### BACKGROUND

Patients with short and longer-term medical needs, as well as aging seniors, require help with housing, personal and medical care. Providing for their personal and medical care needs becomes challenging for families and they often require additional help. In some instances, the individual requiring the personal and medical care lives alone and does not have anyone to help them on a daily basis. Regardless of the support environment, the need for personal and medical care is often best achieved by either hired help, trained caregiver visits to the house, or by admitting the individual to a facility that is equipped to provide such type of personal and medical care.

The growing market of individuals that require housing, personal and medical care has given rise to numerous care agencies, nursing homes, rehabilitation centers, senior living facilities, and other types of care institutions.

Although several options are available to provide the care, several individuals that need such care end up make the wrong or incorrect choice that does not fit their lifestyle or their special needs, circumstances, and preferences. Unable to hop from one care facility to another, either the individuals just stick to the one they have picked and be miserable or when possible, switch to another care facility to discover that the new care facility lacks in other things that are important to the individual.

The problem exists at the outset when an individual attempt to select a caregiver or a care facility. The current methods of searching for the caregiver or the care facility are similar to the same old methods of using a printed yellow page that does not provide you any insight or intelligence into the screening process and instead just leaves it to the user to use guessing methods.

Other alternatives to the yellow page approach are to search for a caregiver or care facility by researching on an Internet Search Engine or on separate ranking portals, such as Yelp. The problem with such searching methods only results in discovering fragmented information that is either misplaced or self-serving and leads the individual to making guesses and wrong choices in attempting to search for appropriate care that matches their preferences and needs.

Yet another method used to search is through the use of a third-party lead generation providers. However, such approach results in inefficiencies and also connects the individual with not properly matched or qualified care givers or care giving facilities. Further, since third-party lead generation providers typically match you with only the caregivers and care giving facilities in their system or those that have a contract with them, their provided recommendations are also self-serving and not well thought out in matching all of the individual's needs and preferences.

Adding to the old methods used, the methods currently available to search for an appropriate caregiver or caregiving facility only allow a zip code search to connect to providers. While the zip code search is useful, it is severely limited and does not address many other patient's needs thereby unable to help consumers determine the right options based on their needs.

Aside from searching and locating an appropriate caregiver or caregiving facility, several other problems exists with the current methods that create obstacles from onboarding the patient to managing continued care management intelligibly.

Further, there is currently no one stop source for educating and matching patients to suitable care providers. Consumers have no transparency on what is available, what are the offerings, and how to find it. Care providers also don't have the tools to leverage prospect data to better serve their patients, keep them satisfied, or prevent patient attrition. This ultimately effects their overall ratings and customer satisfaction that may impact their business.

As such, there exists a real need to determine the preferences and personal and medical needs of a consumer/patient/individual and dynamically use such information to match them with an appropriate caregiver and/or a caregiving facility. There exists a need to provide a complete one stop solution that allows functionality to make onboarding and care management using computerized tools easier. There also exists a need to allow patients/consumers to drive their own search experience and make an informed decision based on data provided.

### SUMMARY

A full end-to-end computerized method and system for matching a caregiver or a care giving facility to a patient is provided, herein after referred to as “Care Matching System,” “Housing and Medical Needs Matching System,” “System,” and/or “end-to-end computerized method and system.” In performing a match, the Housing and Care Matching System uses, among other things, input from the patient, such as patient needs, requirements, and preferences, sophisticated decision trees, real-time data from caregivers and caregiving facilitates, crowd sourced data, scoring mechanisms, and machine learning. In addition to matching a caregiver or a care giving facility to a patient, the Care Matching System provides features that manage patient onboarding, medical insurance verification, paperwork completion and submission. Further the Care Matching System provides tools for managing continued care through use of a decision matrix and deep learning.

The Care Matching System uses both desktop and mobile applications. The desktop version uses a wizard for matching a caregiver or a care giving facility to a patient. It does so by using the Internet and online connections thereby allowing a patient to input data and navigate through the results presented.

The mobile application can be downloaded onto a mobile phone and stored in the memory of the patient's mobile phone. The mobile device is communicatively coupled to the Care Matching System' server. The mobile application provides secure access to the server for performing retrieval and storage of patient information in accordance with HIPAA compliance.

The patient can use the mobile application or the desktop version to provide input to relevant questions asked by the system. The processor in the mobile phone or the processor in the desktop computer communicates with the Care Matching System' processor to step-by-step process and build a patient profile and evaluate patient needs. Answers to various evaluation questions, medical diagnosis, and medical history may be provided by the patient that are recorded and stored into the memory of the Care Matching System.

The backend for both the Care Matching System utilizes complex analysis, decision trees, scoring mechanisms, along with real-time and crowd sourced data to analyze the data stored provided by the patient and stored in its memory and presents matched options to the patient. The selection by the patient launches tools to verify medical insurance and complete the onboarding process along with all requisite paperwork. Tools also provided for direct communication with the facility or scheduling of a tour.

After performing all the analyses, calculations, and curation of care giving facility data. Along with applying deep learning mechanism, the Care Matching System displays the results of the matched care giving facilities on the display screen of the user's electronic device, such as a mobile phone or laptop.

The Care Matching System also allows the patient to input their condition, their day-to-day living needs, and there personal and medical care needs. The Care Matching System analyses the input and recommends one or more types of care for the patient along with specific caregivers and caregiving facilities that provide the recommended care.

### BRIEF DESCRIPTION OF DRAWINGS

The foregoing summary, as well as the following detailed description, will be better understood when read in conjunction with the appended drawings. For the purpose of illustrating the invention, there are shown in the drawings examples that are presently preferred. It should be understood, however, that the invention is not limited to the precise arrangements and instrumentalities shown. In the drawings:

FIG. 1 illustrates an exemplary Care Matching System according to an embodiment of the disclosed subject matter;

FIG. 2 illustrates a computing device for use in the system of FIG. 1 according to the disclosed embodiments;

FIG. 3 is a block diagram illustrating a list of care options provided to the patient according to an embodiment of the disclosed subject matter;

FIG. 4 is a block diagram illustrating a list of housing options provided to the patient according to an embodiment of the disclosed subject matter;

FIG. 5 is a block diagram illustrating a list of personal care options provided to the patient according to an embodiment of the disclosed subject matter;

FIG. 6 is a flowchart illustrating a method of matching a patient to a care giving facility according to an embodiment of the disclosed subject matter;

FIG. 7 is a flowchart illustrating one cycle of operation from beginning to end for matching and admitting a patient to a care giving facility according to an embodiment of the disclosed subject matter;

FIG. 8 is a block diagram illustrating a list of primary medical needs of a patient according to an embodiment of the disclosed subject matter;

FIG. 9 is a block diagram illustrating a list of top concerns relating to the patient's medical care according to an embodiment of the disclosed subject matter;

FIG. 10 is a block diagram illustrating a list of patient's lifestyle interests according to an embodiment of the disclosed subject matter;

FIG. 11 is a block diagram illustrating a list of patient's requirements according to an embodiment of the disclosed subject matter;

FIG. 12 is a block diagram illustrating a list of insurances that are to be selected by the patient according to an embodiment of the disclosed subject matter;

FIG. 13 depicts the results of the analysis performed by the Care Matching System according to an embodiment of the disclosed subject matter;

FIG. 14 depicts a method for patient's insurance verification according to an embodiment of the disclosed subject matter; and

FIG. 15 depicts a crowd sourced network within a city according to an embodiment of the disclosed subject matter.

### DETAILED DESCRIPTION

Reference will now be made in detail to the present examples of the invention illustrated in the accompanying drawings. Wherever possible, the same reference numbers will be used throughout the drawings to refer to the same or like elements.

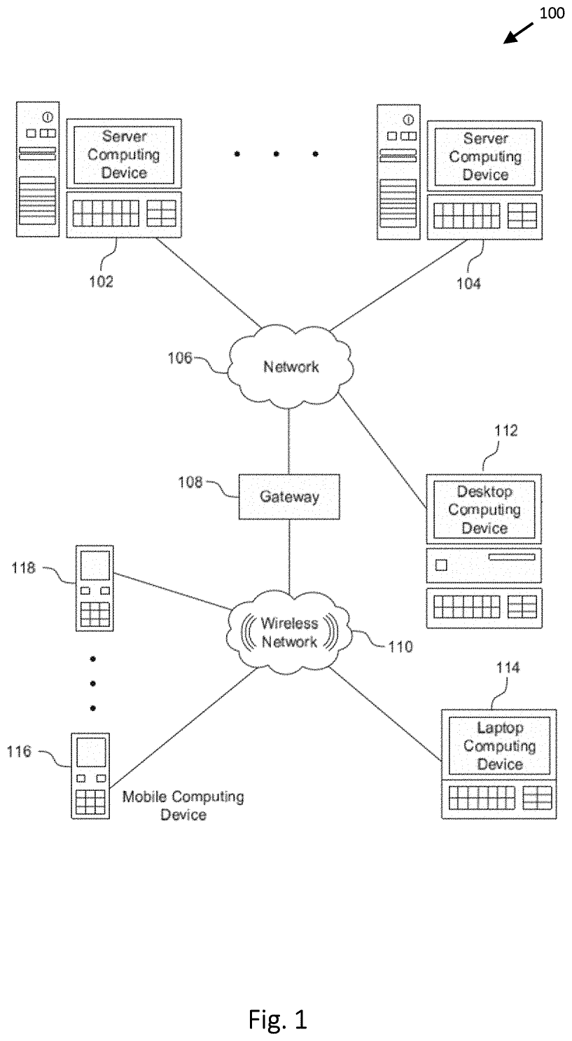

FIG. 1 illustrates an exemplary Care Matching System according to an embodiment of the disclosed subject matter. The disclosure of FIG. 1 is shown for clarity and may include any of these additional components to perform the functionality disclosed herein. Although the system is referred to as Care Matching System, it is not so limited. The nomenclature is simply to provide a name for the system. The system is broad to cover all types of care that are described in this patent application, such as personal care, housing services, memory care, nursing, rehabilitation, specialized care, and care relating to both medical and non-medical needs.

System 100 may include local area networks (LAN) and wide area network (WAN) shown as network 106 and wireless network 110. Gateway 108 is configured to connect remote or different types of networks together, as well as client computing devices 112-118 and server computing devices 102-104.

Client computing devices 112-118 may include any device capable of receiving and sending data over a network, such as wireless network 110. Devices 112-118 may include portable devices such as cellular telephones, smart phones, radio frequency-enabled devices, personal digital assistants, handheld computers, tablets, laptop computers, wearable computers and the like. Devices 112-118 also may include any computing device that connects to a network using a wired communications medium such as personal computers, multiprocessor systems, microprocessor-based or programmable consumer electronics, network personal computers and the like.

Client computing devices 112-118 also may be web-enabled client devices that include a browser application configured to receive and to send web pages, web-based messages and the like. The browser application may be configured to receive and display graphic, text, multimedia, or the like, employing virtually any web-based language, including a wireless application protocol messages (WAP), or the like. In one embodiment, the browser application may be enabled to employ one or more of Handheld Device Markup Language (HDML), Wireless Markup Language (WML), WML Script, JavaScript, Standard Generalized Markup Language (SMGL), Hyper Text Markup Language (HTML), eXtensible Markup Language (XML), or the like, to display and send information.

Client computing devices 112-118 also may include at least one other client application that is configured to receive content from another computing device, including, without limit, server computing devices 102-104. The client application may include a capability to provide and receive textual content, multimedia information, or the like. The client application may further provide information that identifies itself, including a type, capability, name, or the like. In one embodiment, client devices 112-118 may uniquely identify themselves through any of a variety of mechanisms, including a phone number, mobile identification number (MIN), an electronic serial number (ESN), mobile device identifier, network address, such as IP (Internet Protocol) address, media access control (MAC) layer identifier, or other identifier. The identifier may be provided in a message, or the like, sent to another computing device.

Client computing devices 112-118 may also be configured to communicate a message, such as through email, short message service (SMS), multimedia message service (MMS), instant messaging (IM) or the like, to another computing device.

Client devices 112-118 may further be configured to include a client application that enables the user to log into a user account that may be managed by another computing device. Such a user account, for example, may be configured to enable the user to receive emails, send/receive IM messages, SMS messages, access selected web pages, download scripts, applications, or a variety of other content, or perform a variety of other actions over a network. Management of messages or otherwise accessing and/or downloading content, may also be performed without logging into the user account. Thus, a user of client devices 112-118 may employ any of a variety of client applications to access content, read web pages, receive/send messages, or the like.

In one embodiment, for example, the user may employ a browser or other client application to access a web page hosted by a Web server implemented as server computing device 102. Messages received by client computing devices 112-118 may be saved in non-volatile memory, such as flash and/or PCM, across communication sessions and/or between power cycles of client computing devices 112-118.

Wireless network 110 may be configured to couple client devices 114-118 to network 106. Wireless network 110 may include any of a variety of wireless sub-networks that may further overlay stand-alone ad-hoc networks, and the like, to provide an infrastructure-oriented connection for client devices 114-118.

Wireless network 110 may further employ a plurality of access technologies including 3rd (3G), 4th (4G), 5th (5G) and the like generation radio access for cellular systems, WLAN, Wireless Router (WR) mesh, and the like. Access technologies such as 3G, 4G, 5G and future access networks may enable wide area coverage for mobile devices, such as client devices 114-118 with various degrees of mobility. For example, wireless network 110 may enable a radio connection through a radio network access such as global system for mobile communication (GSM), general packet radio services (GPRS), enhanced data GSM environment (EDGE), WEDGE, Bluetooth, high speed downlink packet access (HSDPA), universal mobile telecommunications system (UMTS), Wi-Fi, Zigbee, wideband code division multiple access (WCDMA), and the like. In essence, wireless network 110 may include virtually any wireless communication mechanism by which information may travel between client devices 102-104 and another computing device, network, and the like.

Network 106 is configured to couple one or more servers depicted in FIG. 1 as server computing devices 102-104 and their respective components with other computing devices, such as client device 112, and through wireless network 110 to client devices 114-118. Network 106 is enabled to employ any form of computer readable media for communicating information from one electronic device to another. Network 106 also may include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. Network 106 may include any communication method by which information may travel between computing devices.

The mobile devices 116-118 may be capable of downloading a mobile application (APP) that communicates with the Care Matching System to exchange patient preferences, needs, and requirements.

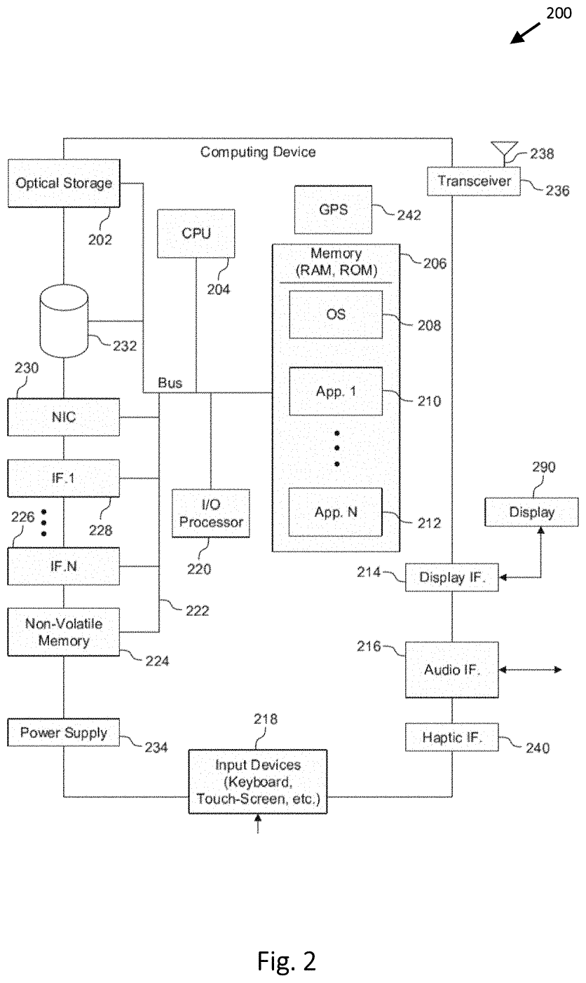

FIG. 2 illustrates a computing device for use in the system of FIG. 1 according to the disclosed embodiments. The computing device 200 configured to execute the functionality disclosed in greater detail below. Computing device 200 may communicate with other devices over system 100 to perform the functions needed for matching a patient to a care provider or a care providing facility. Computing device 200 may be representative of any of the computing devices shown in FIG. 1.

Computing device 200 includes optical storage 202, central processing unit (CPU) 204, memory module 206, display interface 214, audio interface 216, input devices 218, input/output (I/O) processor 220, bus 222, non-volatile memory 224, various other interfaces 226-228, network interface card (NIC) 230, hard disk 232, power supply 234, transceiver 236, antenna 238, haptic interface 240, and global positioning system (GPS) unit 242. Memory module 206 may include software such as operating system (OS) 208, and a variety of software application programs and/or software modules/components 210-212. Such software modules and components may be stand-alone application software or be components, such as DLL (Dynamic Link Library) of larger application software.

Computing device 200 may also include other components not shown in FIG. 2. For example, computing device 200 may further include an illuminator (for example, a light), graphic interface, and portable storage media such as USB drives. Computing device 200 may also include other processing units, such as a math co-processor, graphics processor/accelerator, and a Digital Signal Processor (DSP).

Central processing unit (CPU) 204 may be the main processor for software program execution in computing device 200. CPU 204 may represent one or more processing units that obtain software instructions from memory module 206 and execute such instructions to carry out computations and/or transfer data between various sources and destinations of data, such as hard disk 232, I/O processor 220, display interface 214, input devices 218, non-volatile memory 224, and the like.

Memory module 206 may include RAM (Random Access Memory), ROM (Read Only Memory), and other storage means, mapped to one addressable memory space. Memory module 206 illustrates one of many types of computer storage media for storage of information such as computer readable instructions, data structures, program modules or other data. Memory module 206 may store a basic input/output system (BIOS) for controlling low-level operation of computing device 200. Memory module 206 may also store OS 208 for controlling the general operation of computing device 200.

Display interface 214 may be coupled with a display unit 290, such as liquid crystal display (LCD), gas plasma, light emitting diode (LED), or any other type of display unit that may be used with computing device 200. Display unit 290 coupled with display interface 214 may also include a touch sensitive screen arranged to receive input from an object such as a stylus or a digit from a human hand. Display interface 214 may further include interface for other visual status indicators, such light emitting diodes (LED), light arrays, and the like. Display interface 214 may include both hardware and software components. For example, display interface 214 may include a graphic accelerator for rendering graphic-intensive outputs on the display unit. In one embodiment, display interface 214 may include software and/or firmware components that work in conjunction with CPU 204 to render graphic output on the display unit. Display may be on the patient's mobile device or on their laptop.

Input devices 218 may include a variety of device types arranged to receive input from a user, such as a keyboard, a keypad, a mouse, a touchpad, a touch-screen (described with respect to display interface 214), a multi-touch screen, a microphone for spoken command input (describe with respect to audio interface 216), and the like.

I/O processor 220 is generally employed to handle transactions and communications with peripheral devices such as mass storage, network, input devices, display, and the like, which couple computing device 200 with the external world. In small, low power computing devices, such as some mobile devices, functions of the I/O processor 220 may be integrated with CPU 204 to reduce hardware cost and complexity. In one embodiment, I/O processor 220 may the primary software interface with all other device and/or hardware interfaces, such as optical storage 202, hard disk 232, interfaces 226-228, display interface 214, audio interface 216, and input devices 218.

An electrical bus 222 internal to computing device 200 may be used to couple various other hardware components, such as CPU 204, memory module 206, I/O processor 220, and the like, to each other for transferring data, instructions, status, and other similar information.

Non-volatile memory 224 may include memory built into computing device 200, or portable storage medium, such as USB drives that may include PCM arrays, flash memory including NOR and NAND flash, pluggable hard drive, and the like. In one embodiment, portable storage medium may behave similarly to a disk drive. In another embodiment, portable storage medium may present an interface different than a disk drive, for example, a read-only interface used for loading/supplying data and/or software.

Hard disk 232 is generally used as a mass storage device for computing device 200. In one embodiment, hard disk 232 may be a Ferro-magnetic stack of one or more disks forming a disk drive embedded in or coupled to computing device 200. Alternatively, hard drive 232 may be implemented as a solid-state device configured to behave as a disk drive, such as a flash-based hard drive. In yet another embodiment, hard drive 232 may be a remote storage accessible over network interface 230 or another interface 226, but acting as a local hard drive.

Transceiver 236 generally represents transmitter/receiver circuits for wired and/or wireless transmission and receipt of electronic data. Transceiver 236 may be a stand-alone module or be integrated with other modules, such as NIC 230. Transceiver 236 may be coupled with one or more antennas for wireless transmission of information.

Antenna 238 is generally used for wireless transmission of information, for example, in conjunction with transceiver 236, NIC 230, and/or GPS 242. Antenna 238 may represent one or more different antennas that may be coupled with different devices and tuned to different carrier frequencies configured to communicate using corresponding protocols and/or networks. Antenna 238 may be of various types, such as omni-directional, dipole, slot, helical, and the like.

Haptic interface 240 is configured to provide tactile feedback to a user of computing device 200. For example, the haptic interface may be employed to vibrate computing device 200, or an input device coupled to computing device 200. For example, when a message is received by device 200 or another event occurs, the device may vibrate to alert the user.

Global positioning system (GPS) unit 242 can determine the physical coordinates of computing device 200 on the surface of the Earth, which typically outputs a location as latitude and longitude values. GPS unit 242 can also employ other geo-positioning mechanisms, including, but not limited to, triangulation, assisted GPS (AGPS), E-OTD, CI, SAI, ETA, BSS or the like, to further determine the physical location of computing device 200 on the surface of the Earth. It is understood that under different conditions, GPS unit 242 can determine a physical location within millimeters for computing device 200. In other cases, the determined physical location may be less precise, such as within a meter or significantly greater distances. In one embodiment, however, a mobile device represented by computing device 200 may, through other components, provide other information that may be employed to determine a physical location of the device, including for example, a MAC address.

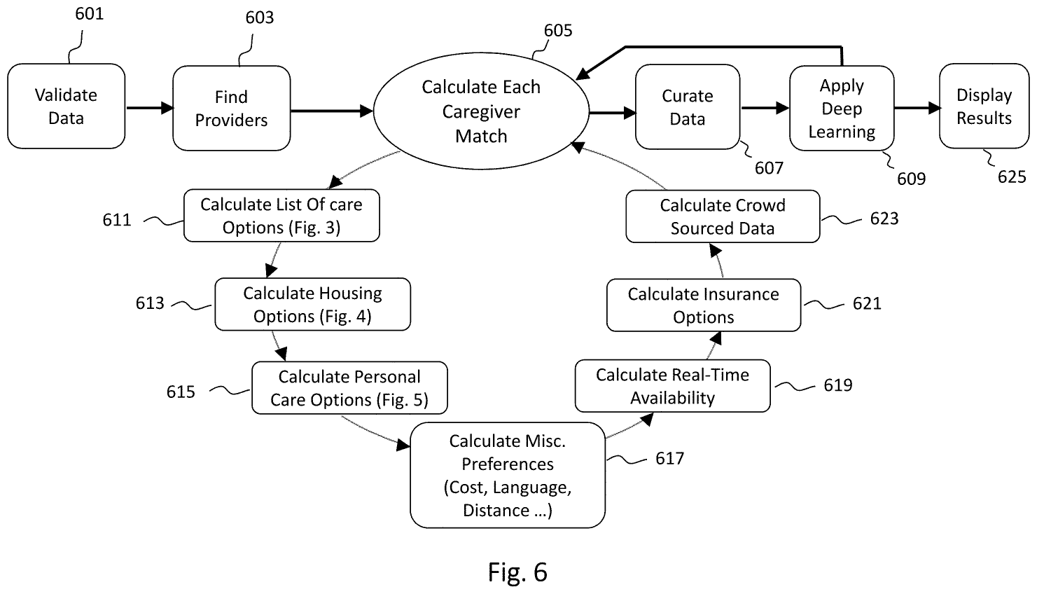

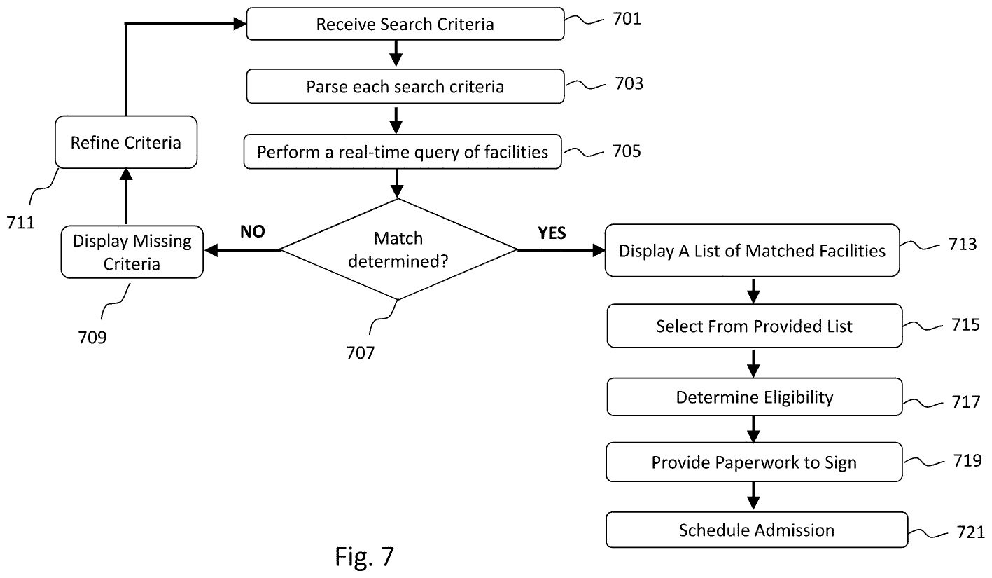

Using instructions stored in memory module 206, device 200 may read the instructions to have CPU 204 execute the functions specified in the instructions. These functions are disclosed in greater detail by FIGS. 6 and 7.

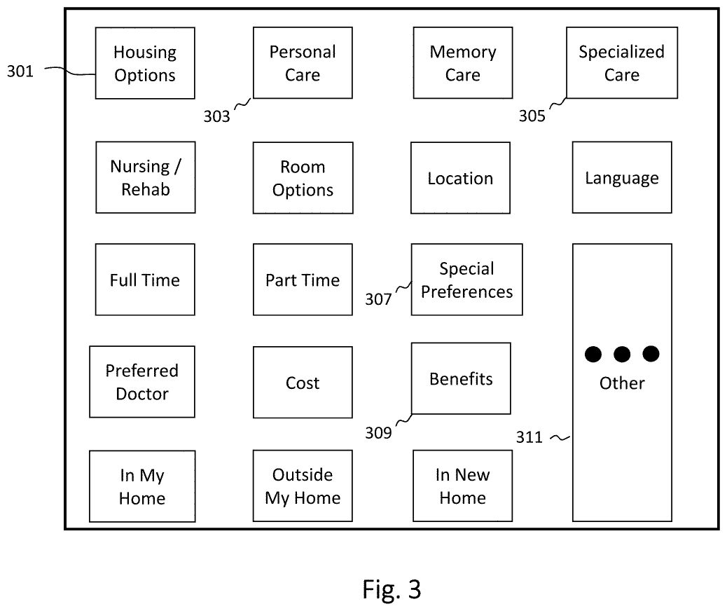

FIG. 3 is a block diagram illustrating a list of care options provided to the patient according to an embodiment of the disclosed subject matter. The processor of the Care Matching System processes a plurality of care options and displays them on the patient's electronic device.

The terms patient, user, care receiver, consumer, resident, prospect, prospective resident, senior, and senior citizen are used synonymously in this patent application and they refer to a person receiving or intended to receive medical treatment or another type of care, such as personal care or memory care.

The terms caregiver, caregiving facility, companies that offer caregiving in the home, caregiving outside the home, senior housing community, nursing home, senior living center, senior care, housing services, rehabilitation center, and agencies that provide caregiving matching are used synonymously in this patent application and they refer to a person, skilled technician, nurse, or a care giving facility that is in the business of providing care to the patient.

The patient may select any one or more of the care options that are relevant to their medical needs. Some of the options presented include: Housing 301, Personal Care 303, Memory Care, Nursing, Rehabilitation, Specialized Care 305, Amount of time required, such as either full time or part time, In My Home, Outside My Home, In New Home, Location, Language, Special Preferences 307, Cost, and/or Benefits 309. In addition to the care options presented, other options 311 may be added or deleted by the Care Matching System based on changing needs.

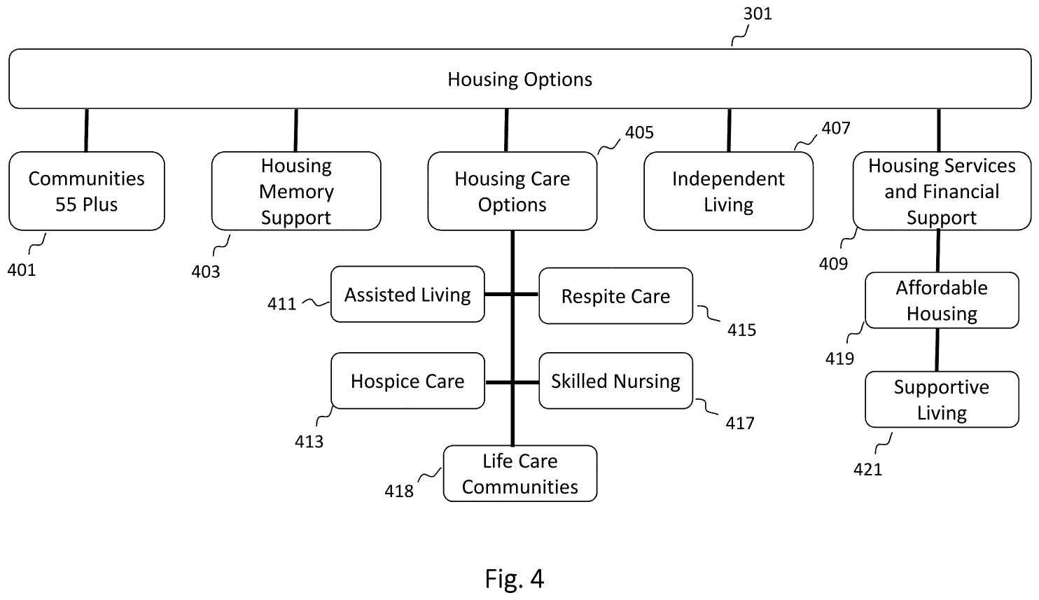

The housing options 301 and personal care options 303 are further defined in FIGS. 3 and 4.

### Memory Care

Memory Care, one of the care options presented by Care Matching System and displayed on the patient's electronic device includes:
a) Home Support Non-Medical—Home Care Agencies
b) Home Support Non-Medical—Continuing Care At Home

The above options are non-medical options. These may include servicing various patient mobility and other daily routine needs that do not require medical attention. As such, the required skill is those of a regular caregiver that do not require special medical certifications or licensing, such as for example a nurse.
c) Home Support Medical—Home Care Agencies
d) Home Support Medical—Continuing Care At Home

The above options are medical options that require a trained and skilled medical staff. These include cases where the patient may need skilled registered nurse (RN) or physical therapy (PT) or occupational therapy (OT) type if care. It may also include medical care for a diabetic patient or a patient that has undergone surgery and needs medical follow up. The type of medical care may also include IV administration, wound care, trach or vent care. The patient may have a chronic condition or be recovering from an acute illness, surgery, accident or a change in medical condition that may require an ongoing either short- or long-term care by a skilled medical person. The patient may decide to get the type of medical care needed by either a home health agency or through continuing care at home.
e) Housing Services and Financial Support—Supportive Living

Housing services and financial support is yet another type of care that is provided by the Care Matching System. A patient that needs to be relocated to a facility or needs financial help to cover their medical and care costs may select this option on their display. The Care Matching System on the backend would process the request and navigate the patient through a set of questions and take that into consideration when recommending a care facility.
f) Housing Memory Support—Memory Care

Yet another type of care offered for selecting through the Care Matching System includes Housing Memory Support—Memory Care. This could be for patient suffering from dementia or Alzheimer's disease and have trouble remember things that they may need on a regular basis, including taking medications on time. This type of care would involve skilled nurses and therapists that have special training, including certifications, in dealing with dementia and Alzheimer's patients.
g) Out of Home Service—Adult Day Care
h) Out of Home Service—Rehab Centers
i) Out of Home Service—Pace Centers
j) Out of Home Service—Palliative Care

Care Matching System may also provide additional out of home services as listed above. The Adult Day care options includes providing for the senior citizen's daily needs, such as meals, activities, transportation, and other hands-on type of needs for their daily living.

Rehab care is another option that provides therapy services, skilled nursing, PT, OT, for patient that are undergoing rehabilitation.

PACE is yet another type of care option provided to the patients for selection if they meet its requirements and have been preapproved for such care. PACE, which stands for stands for Programs of All-Inclusive Care for the Elderly, is a Medicare and. Medicaid government program that helps elderly and others needing medical attention meet their health care needs while residing either at home of in the local community instead of going to a care facility.

Palliative care is yet another care option provided for selection by the Care Matching System. Palliative care is basically interim care for patients that that are neither home bound nor ready to be admitted to a long-term care facility of hospice. A palliative care team is a skilled medical and multidisciplinary team that works with the patient and their family to provide medical, social, and daily needs support and keep them from unnecessarily visiting an emergency room (ER) by handing their medical needs. The skilled personnel, such as palliative care specialists, doctors and nurses, as well as social workers provide in-home care for care that is typically given at hospitals, nursing homes, or clinics. It is covered by both Medicare and Medicaid and may also have to paid out of pocket or through personal insurance, it all depends on the patient's insurance and their finances.

### Specialized Care

The Care Matching System may also provide a specialized care 305 option for the patient to select. The patient may use their electronic device and provide input through this option that is then processed by the processor of the Care Matching System and scored and considered in presenting a matching recommendation. This is care for a specialized situation that is not common. For example, care for a patient having a rare disease or a patient with special handicap situation is offered through specialized care 305.

### Nursing/Rehabilitation

The Care Matching System also offers a nursing/rehabilitation care option to the patient for selection. Through this type of care, a specialized rehabilitation nurse assist individual with a disability and/or chronic illness perform their daily functions. In cases, where the patient can no longer return to their original state, the rehabilitation nurses help adapt and maintain an altered lifestyle that would still help them provide for their daily needs. Some the sub-choices that may be offered through this type of care include:
a) Home Support Medical—Home Care Agencies
b) Home Support Medical—Continuing Care At Home
c) Housing Care Options—Respite Care
d) Housing Care Options—Hospice Care
e) Housing Care Options—Skilled Nursing
f) Housing Care Options—Life Care Communities
g) Housing Care Options—Palliative Care
h) Out of Home Service—Rehab Centers
i) Out of Home Service—Pace Centers
j) Out of Home Service—Palliative Care

### Full or Part Time Care

Depending on the type of medical needs and the treatment plan from a doctor, patients may need either long term or short-term care. The Care Matching System allows a patient to customize the type of time commitment to their liking or to the treatment plan.
Room Options

The Care Matching System may also provide a care option for the patient to select their room. The patient may use their electronic device and provide input through this option that is then processed by the processor of the Care Matching System and scored and considered in presenting a matching recommendation.

The current and old methods allow a healthcare facility to assign a room for an incoming patient without much consideration for their personal needs. Usually whatever room is available gets assigned. In many instances, the room type and the look and feel does not suit the patient and either they are unhappy, depressed, or leave for another facility. In one embodiment of the present invention, through use of this option, the patient may provide input such as brightness of a room, location of the room within the facility, noise level near the room, room size, and other preferences that are important to them in living in that room for either a short or long term.
Location

The Care Matching System may also provide a care option for the patient to select their location. The patient may use their electronic device and provide input through this option that is then processed by the processor of the Care Matching System and scored and considered in presenting a matching recommendation.

The patient may select this option and provide a location, such as a city or zip code, and a mile radius within which they prefer to find their care giving facility. If a patient wants to reside close to their family, or a place of interest, this option can be selected in finding their care facility. For example, a patient may limit their search for a matched care facility to be within 25 miles of their home or their family's home. They may also want to choose to be close to a church they have been going to all their life.

This location and miles option may also be used to find a care giver that lives closer to their home. Since a caregiver that lives closer could be more dependable and reliable, or able to make last minute runs as needs, a patient may prefer for their comfort to select a local caregiver that can service the patient regularly.

### Language

The Care Matching System may also the patient to select their language, preferred doctor, or preferred nurse. Depending on availability of the preferred doctor or nurse or a caregiver that understands their language, the Care Matching System would process the request and considered in presenting a matching recommendation.

### Special Preferences

The Care Matching System may also allow the patient to input their special preference. The request is then processed by the processor of the Care Matching System and scored and considered in presenting a matching recommendation.

Special preferences are different from special medical needs. For example, these may include an ethnic meal, a certain type of environment, specialized facilities that cater to a specific ethnic, religious, or military group etc.

### Cost

The Care Matching System allows the patient to select a budget. The patient may use their electronic device and provide input through this option that is then processed by the processor of the Care Matching System and scored and considered in presenting a matching recommendation.

In one embodiment of the present invention, the Care Matching System allows a patient to select from a group of preselected options as listed below.
a) $0-$2500
b) $2500-$5000
c) $5000-$8000
d) $8000 and above
e) Customized Option

Using the above options, the patient can manage their monthly budget. The patient can also customize their budget and input an option that works for them to determine if a care facility can work with them on their budget or get them additional assistance.

The Care Matching System ensures that the selected budget, including the customized option, is matched with a care provider or care facility that can work within the budget and provides names and listing of such care providers and care facilities to the patient.

### Benefits

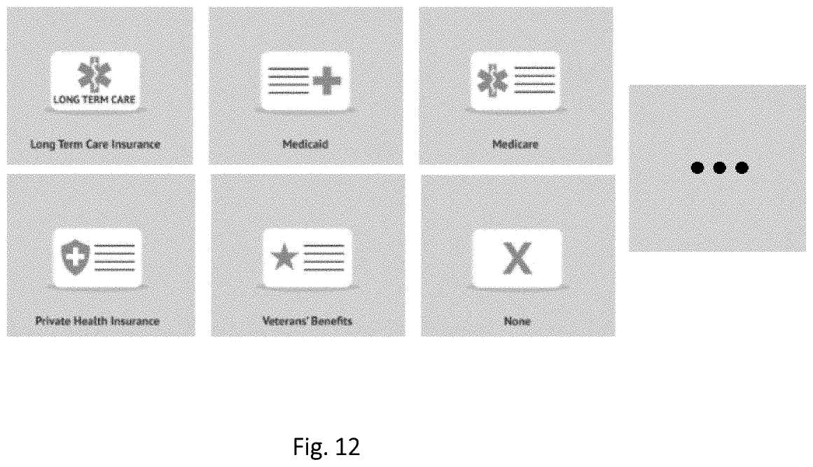

Benefits are described in more detail in FIG. 12. In brief, the Care Matching System allows the patient to input their medical insurance information in an attempt to match a care facility, or a care giver, that takes their medical insurance. Additional options, such as insurance verification and budget management tools are also provided and will be discussed in the figures below.

### In My Home/Outside My Home

The patient may also provide an input using their electronic device as to whether they prefer in or out of home care. The input is then processed by the processor of the Medical Needs Matching and used in considering a match.

### Additional Options

Although several types of care options and their sub-options have been defined above, the present invention is not so limited. Additional customized selections may be offered by the Medical Needs Matching or a patient may suggest a customized option that will be considered and added to the list mentioned above.

FIG. 4 is a block diagram illustrating a list of housing options provided to the patient according to an embodiment of the disclosed subject matter. These housing options allow a patient to customize their living preferences at a care facility. The Care Matching System may allow the patient to input there from a list of predefined housing options using their electronic device. Although a certain number of housing options are provided, the invention is not so limited and other options may be added later.

Once the patient selects their housing option, the request is then processed by the processor of the Care Matching System. Several sub-options may also be presented, and their selection is also processed. All the housing selections made by the patient are then scored and considered in presenting a matching recommendation.

In one embodiment, the Care Matching System presents the following housing options:
a) Community 55 Plus
b) Housing Memory Support
c) Housing Care Options
d) Independent Living
e) Housing Services and Financial Support

Some of the options provided here are described earlier in FIG. 3. Those not described in FIG. 3 are described below,

### Community 55 Plus

As a sub-option of the housing option 301, in one embodiment, the Care Matching System allows a patient to select the Community 55 Plus option. The prospect or prospective resident may use their electronic device and provide input through this option that is then processed by the processor of the Care Matching System.

The Community 55 Plus option allows patients that are 55 years or older to live in a planned community with a variety of amenities that cater to the senior resident. The residents that have common interests and wanting to live with other likeminded 55-year old's chose this option. Some may have an active and healthy lifestyle and others may want more quieter surroundings and a mo carefree lifestyle.

### Housing Memory Support

As described in FIG. 3, this option is for patients suffering from dementia or Alzheimer's disease. The housing would be suited to specially cater to such individuals. For example, in addition to having a secured environment and skilled staff that can handle dementia or Alzheimer's disease, the facility may have activities and engagement programs that cater to them.

### Housing Care Options

As a sub-option of the housing option 301, in one embodiment, the Care Matching System allows a patient to select from one of many housing care options 405. These sub-options include:
a) Assisted Living 411
b) Hospice Care 413
c) Respite Care 415
d) Skilled Nursing 417
e) Life Care Communities
f) Additional Housing Care Options

The patient may use their electronic device and provide input to select a sub-option that is then processed by the processor of the Care Matching System to determine a match with a care facility. Although a certain number of housing care options are provided, the invention is not so limited and other options may be added later.

Assisted living 411 option is part of long-term care that combines housing, personal care, and addressing of medical and physical needs for individuals who need assistance with normal daily activities. There are specially designed facilities that cater to assisted living.

Hospice care 413 is a s specialized type of care for those patients facing a terminal illness or a life-limiting illness. The care addresses the patient's pain management as well as helps the patient with their physical, emotional, and spiritual needs.

Respite care 415 is a temporary relief service that helps a caregiver for a short period of time, such as for example, if the primary caregiver is busy, out of town, ill, or unable to attend to the patient. This gives the primary caregiver some time for themselves and is a customized type of care. In some instances, it can also be care for just the night time that allows the primary caregiver to sleep while someone else is attending to the patient at night.

Skilled nursing 417 and life care communities 418 are also among the choices offered by the Care Matching System.

### Independent Living

Independent living 407 is also a sub-option of the housing option 301 that is offered by the Care Matching System. A patient desiring to live independently yet receive care on their own through a home care option may chose this option.

### Housing Services and Financial Support

The housing services and financial support sub option 409 is a sub-option of housing that may be selected by the patient. This option is further divided into affordable care 419 and supportive living 421. These options allow the patient to receive aid and support both for their housing as well as medical needs.

Although various housing options and their sub-options were described, the invention is not so limited and additional housing options can be added to the Care Matching System. These may also be some combinations and hybrid variation of the options and sub-options presented.

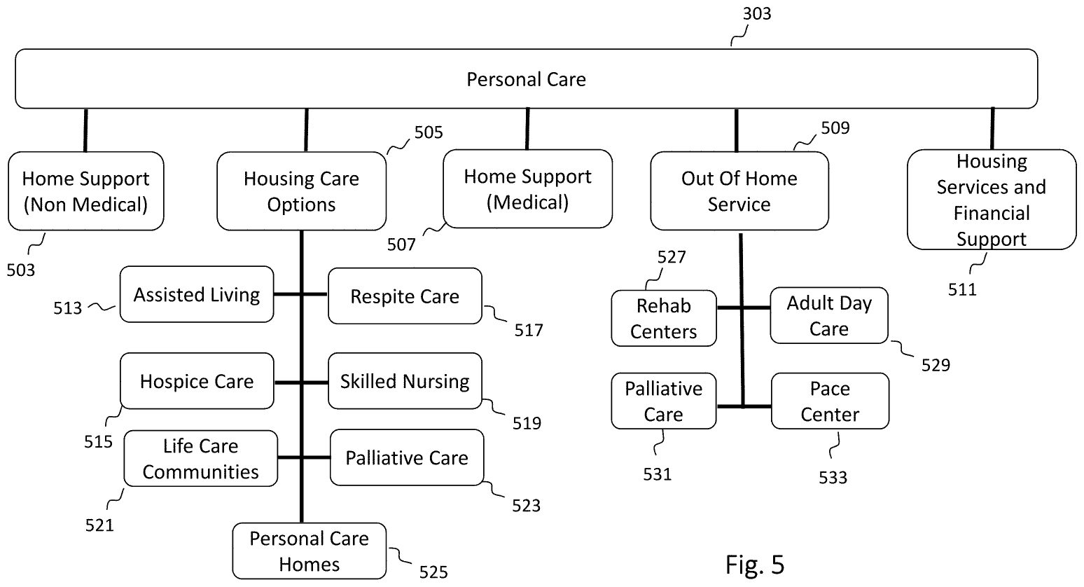

FIG. 5 is a block diagram illustrating a list of personal care options provided to the patient according to an embodiment of the disclosed subject matter.

The options presented by the Care Matching System under this category allow a patient to customize their personal care either at home or in a care facility. As mentioned earlier, Personal Care is care that is provided to patients with special needs. Typically, it is provided at the patient's home but can also be provided at a care facility. Individuals that are senior, getting older, disabled, chronically ill, prolonged illness, have physical or developmental disabilities, recovering from surgery, or disabled often require personal care.

The duties for personal care include helping with day to day needs such as bathing, washing your hair, getting dressed, eating food, or simply maneuvering around the house or being transported to a place. At times, personal care also includes companionship or emotional support. Personal care workers help the patient with all such needs so they can live a normal life while getting the personal care.

Personal care may be covered by Medicare and Medicaid but in many instances is also an out of pocket cost to the patient. Understudying the budget and customizing the care is very important and can be selected by the patient or their family member through the Care Matching System.

Several predefined options are presented on the patient's display screen that are processed on the back end by the processor of the Care Matching System upon selection from the patient. The invention is not limited to these predefined personal care options and other personal care options may be added later. Once the patient selects their personal care options, the processor scores their preferences and analyzes them to present a matching recommendation.

In one embodiment, the personal care options, some of which are similar to the housing options in FIG. 4, include the following:
a) Home support 503
b) Housing Care Options 505 (Includes assisted living 513, hospice care 515, respite care 517, skilled nursing 519, life care communities 521, palliative care 523, and personal care homes 525)
c) Home support 507
d) Out of Home 509 (Includes rehab centers 527, adult day care 529, palliative care 531, and pace centers 533)
e) Housing Services and Financial Support 511

These predefined options, and their sub-options have been described in earlier figures, as depicted in FIG. 5.

FIG. 6 is a flowchart illustrating a method of matching a patient to a care giving facility according to an embodiment of the disclosed subject matter. The method is implemented by the Care Matching System as shown in FIGS. 1 and 2. The effectiveness of any match between a care receiver/patient and a caregiver or caregiving facility depends upon, at a minimum, the requirements defined by the patient, the crowdsourced data and their experiences, and real-time insight into the caregiver or caregiving facility's availability, their offerings and their skill sets. The effectiveness is further enhanced by applying deep learning mechanisms and improving the data set periodically after having additional data points relating to both the patient side as well as the caregiver side. As a result, the deep learning mechanisms, along with decision tree analysis tools, scoring mechanisms, real-time caregiver data, and crowdsourced data, together analyze data and navigate through complex alternatives to provide a result that matches the patient's desires and needs.

In one embodiment of the invention, the method of matching a patient to a care giving facility depicted in FIG. 6 performs the following steps below. At step 601, the method validates data entered by the patient or a patient representative. The validation is to ensure that proper data is entered that is error free. In some case, when a keyword is entered that is not recognized by the system, then the Care Matching System may provide suggestions or alternatives.

At step 603, the method uses the Care Matching System to find care providers. These may be the list of all care providers based on the initial data entered or a list of providers within a vicinity or based on some other system selected criteria.

At step 605, an analysis is performed by the processor of the Care Matching System to match a plurality of criterion. The criterion used are defined in steps 611-623.

At step 607, after all the calculations in steps 611-623 are performed, analyzed, and parsed through a logic decision tree, the data is curated to slice and dice and rank the data in terms of the highest percentage of match. For example, the ranking for a care facility that is 86% will be displayed in a higher matching order than a care giver facility that is a 73% match.

At step 609, the data is compared with prior iterations of the method as well as other data gathered through crowdsourcing. Since each cycle of method from steps 601-623 are stored, the deep learning algorithms use each cycle to further enhance and curate the data for subsequent cycles. In performing such curation, Steps 605 and its sub steps 611-623 may be repeated. In one embodiment, the deep learning process uses a neural networks model for analyzing data sets. The model allows intelligent enhancing of data sets based on relevancy from a prior data set.

At step 625, the final results are displayed on the display screen of the patient's electronic device.

As mentioned above steps 611-623 are performed to calculate and analyze each set of patient requirements, needs, and preferences as they relate to care options. At step 611, the method uses the processor of the Care Matching System to analyze and calculate the list of care options as shown in FIG. 3. The analysis consists of comparing the patient given requirements, needs, and preferences for care options with the care facility's offerings, skills, and availability for the requirements, needs, and preferences entered by the patient. In one embodiment, a score is assigned if all the criteria are met. In another embodiment, a percentage of score is assigned if some of the criteria relating to care options are met. In yet another embodiment, higher relevant requirements are weighted higher than preferences.

At step 613, the method uses the processor of the Care Matching System to analyze and calculate the list of housing options as shown in FIG. 4. The analysis consists of comparing the patient given requirements, needs, and preferences for housing options with the care facility's offerings, skills, and availability for the requirements, needs, and preferences entered by the patient. In one embodiment, a score is assigned if all the criteria are met. In another embodiment, a percentage of score is assigned if some of the criteria relating to housing are met. In yet another embodiment, higher relevant requirements are weighted higher than preferences. For example, a handicapped person will require a handicapped accessible room and shower and they may also prefer a room that has a outdoor view. In such a situation, since handicap accessibility is a need, and not a preference, it will be weighted more than the view in calculating the overall housing score.

At step 615, the method uses the processor of the Care Matching System to analyze and calculate the list of personal care options as shown in FIG. 5. The analysis consists of comparing the patient given requirements, needs, and preferences for personal care with the care facility's offerings, skills, and availability for the requirements, needs, and preferences entered by the patient. In one embodiment, a score is assigned if all the criteria are met. In another embodiment, a percentage of score is assigned if some of the criteria relating to personal care are met. In yet another embodiment, higher relevant requirements are weighted higher than preferences.

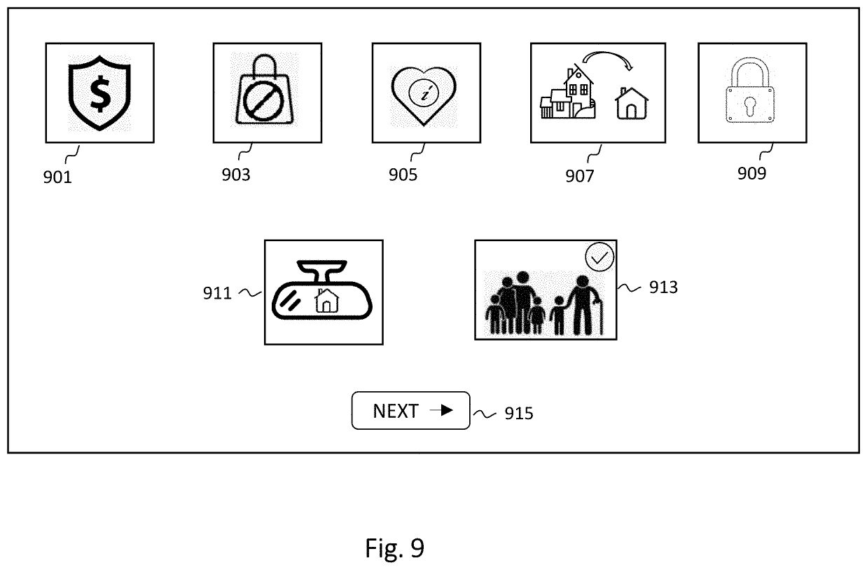

At step 617, the method uses the processor of the Care Matching System to analyze and calculate miscellaneous requirements, needs, and preferences entered by the patient. These may be items such as cost, language, distance from home or a family member, look/feel of the room, specific type of community, such as an ethnic community, type of food served, and many other items that may be either preselected from the offerings or defined by the patient. Some of the miscellaneous options are depicted in FIGS. 9-11. The analysis consists of comparing these patients defined requirements, needs, and preferences with the care facility's offerings, skills, and availability. As mentioned earlier, a score may be assigned.

At step 619, the method uses the processor of the Care Matching System to analyze and calculate the real time availability of requested items at the care facility. For example, the Care Matching System communicates with the care facility in real-time to determine if a certain type of room, bed, or patient listed need and preference is available. Even though the facility's offerings and skills may be matched with those requested by the patient, if the offerings and skills are not available due to the capacity being full or some other reason, such as the skilled person is on vacation, or the desired type of room is occupied, then the patient's need is not fully addressed. As such the Care Matching System communicates in real-time with the care giver facility and determines the availability along with a schedule and future availability if currently booked. As mentioned above, a score is assigned based on current and future availability that matches the patient's criteria.

At step 621, the method uses the processor of the Care Matching System to analyze and calculate the insurance or benefits options as shown in FIG. 12. The analysis consists of comparing the patient insurance to the insurance accepted by the care giving facility. A score may be assigned based on the match determined.

At Step 623, the method uses the processor of the Care Matching System to analyze crowdsourced data. This includes data provided by other patients and care facilities, along with reviews or types of care, offerings, and customer satisfaction. The Care Matching System takes these scores into account when calculating and presenting a recommendation to the patient. For example, if the patient is specific on their need to have a firm bed, and the reviews and crowdsourced data has some data relating to firm bed, then such data is taken into account when scoring and ranking the choices.

FIG. 7 is a flowchart illustrating one cycle of operation from beginning to end for matching and admitting a patient to a care giving facility according to an embodiment of the disclosed subject matter. The method is implemented by the Care Matching System as shown in FIGS. 1 and 2.

At step 701, the Care Matching System receives search criteria from a patient. The patient may be using a secure mobile application (APP) 210, 212 downloaded on their electronic device or the internet to connect with the Care Matching System and input their criteria. They may also be using a Wizard that is part of the Care Matching System and provided through the Internet online to securely login and enter patient details. Either the patient or a patient representative may enter patient needs, requirements, and preferences. The input date is stored in the memory 224 by the processor 220.

At step 703, the Care Matching System parses all the entered and received data in step 701. In one embodiment the parsing consists of compartmentalizing the data into separate groups, such as housing, personal care, location, insurance etc.

At step 605, the Care Matching System performs a query of all the care facilities within a geographical area to determine a match. In another embodiment, the query may be in real-time.

At step 707, the Care Matching System determines if the entered criteria in Step 701 matches with the offering and skills of a care facility. If there is no match, then at Step 709, the Care Matching System displays a list of one or more criteria's that are not available at the care facility. The patient at Step 711 may refine, change, or narrow the search criteria to perform another search. The Care Matching System repeats steps 701-707 until a match is found. In another embodiment, the Care Matching System allows a preset number of searches before concluding that a match cannot be found.

If it is determined that at step 707 that a match is made, then the Care Matching System at step 715 displays a list of all matched care facilities on the user's electronic device. The Care Matching System may also allow the patient to sort, filter, and arrange the list of provided care facilities using their APP features on their mobile phone or the laptop tools.

At step 715, the user may select from the displayed list of care facilities. The patient/user may also tag the facilities as favorites or rank them for later viewing.

At step 717, the Care Matching System determines insurance eligibility based on the car facility selected. Since insurance payout, cost and budgeting, and amount of insurance coverage is critical in determining admission, the Care Matching System determines if the selected facility as well as the needs, requirements, and preferences selected are covered by the patient's medical insurance. More details are provided in FIG. 12.

At Step 719, all the insurance, legal, and administrative paperwork required to complete admission can be reviewed and signed by the patient thereby reducing onboarding time as well as confusion or misunderstanding whether the care facility selected takes the insurance provided.

At Step 712, the Care Matching System provides options for both the care facility and the patient to coordinate schedule and schedule an admission date and time. In case where the availability is in the future, the Care Matching System calendars an admission date and sends reminders and updates on the patient's mobile App. In another embodiment, the Care Matching System also schedules a tour prior to admission.

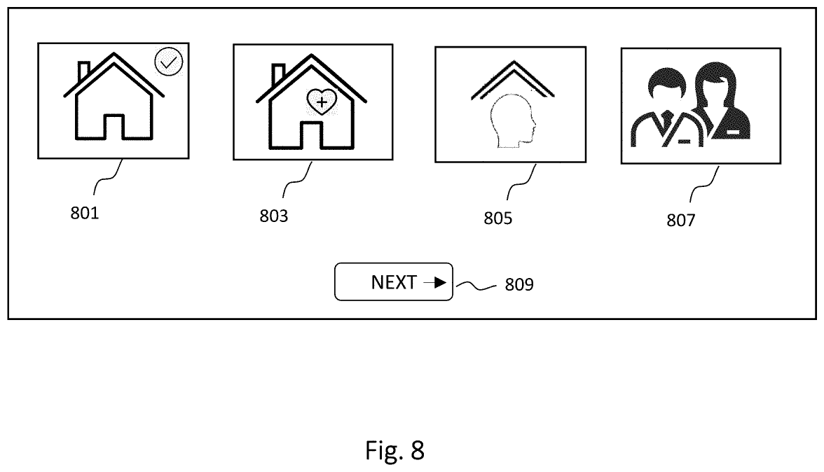

FIG. 8 is a block diagram illustrating a list of primary medical needs of a patient according to an embodiment of the disclosed subject matter. As mentioned earlier in FIGS. 3-5, the Care Matching System can be displayed to the patient one of several platforms, e.g. mobile application, desktop version, thought internet connect to an interactive wizard online. In this embodiment, the user interface of the wizard, alternatively the mobile application is depicted. At shown, 801 represents housing options, 803 represents personal care and support options, 805 represents memory care options, and 807 represents nursing, rehabilitation, and end of life, such as hospice options. The user interface allows the patient to select any one or more of the options 801-807 to then proceed to the next step 809.

In one embodiment, the patient selects either part time or full-time option as a next step after selecting the options in FIG. 8 (now shown in figures). The selection allows the Care Matching System to analyze care providers and care providing facilities based on the type of part time or full-time care provided.

Further, as a next step (not shown in figures), the user interface may provide the choices of In My Home, Outside My Home, or In a New Home that can be selected by the patient.

FIG. 9 is a block diagram illustrating a list of top concerns relating to the patient's medical care according to an embodiment of the disclosed subject matter. The user interface connected to the Care Matching System may a list of these top concerns on the wizard (as well as on the mobile APP and desktop application). The displayed items include asset protection 901, not being a burden 903, available care 905, moving/downsizing 907, safety and security 909, leaving my home 911, and proximity to family 913. The user may select any one or more of these items and select next 915 to proceed to the next screen.

Some of terms listed were defined above. In brief, asset protection 901 relates to a patient having a concern to secure their home and assets and therefore wanting to securely store them somewhere or be close to them. The not being a burden 903 option relates to the patient who may either be living with a relative or close to a relative making sure that although some help from the relative is acceptable, perhaps even some financial assistance, but they want to be more independent and be a burden.

The available care 905 option explores the available care offering of the care giver/facility. These may be specific types of care that are available that may be distinct from one facility to another.

The moving/downsizing 907 option relates to a patient that may have been living in a bigger home and now due to their medical condition, or some other criteria is downsizing to a smaller home. The concerns may relate to making sure the new space is helpful in moving around and addressing their daily living needs.

Safety and security 909 concern relates to the neighborhoods where the patient feels safe. It may also deal with a patient that is affluent or well known in the community requiring additional security and safety. Other options, leaving my home 911, and proximity to family 913 relates to location, distance and features offered by the care facility that were originally at home.

Although not shown, as a next step, the user interface connected to the Care Matching System may display a field for the patient to enter their zip code, city, and a radius in miles within which they wish to find the care provider or facility. The user interface may also ask if the search is being performed by the patient or their relative.

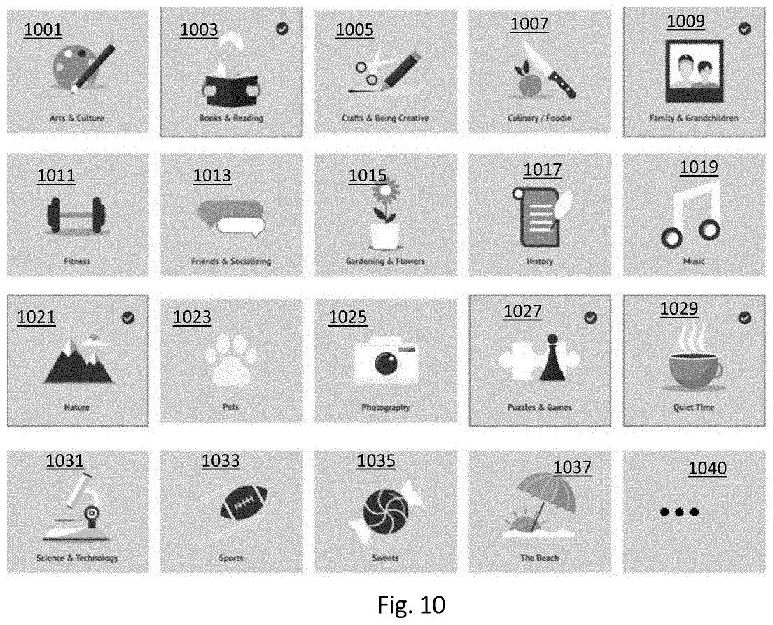

FIG. 10 is a block diagram illustrating a list of patient's lifestyle interests according to an embodiment of the disclosed subject matter. The user interface connected to the Care Matching System may display a large list of interests 1001-1037. Additional interest 1040 may be added over time by the system or suggested by the patient.

The patient may select one of more of the interest. For example, in FIG. 10, the patient has selected 5 interests (1003, 1009, 1021, 1027, and 1029). All the interest selected and matched by the care giving facility will be scored. The weighing for each interest may also be defined as low, medium, or high.

The Care Matching System may also recognize inconsistencies in the selection and adjust or score them accordingly. For example, a patient who selects quite time, nature, and reading - which relate to quieter and calmer options—and then also selects socializing, sports, and large gathering—which relate to a more active rather than calm and quiet options, may be somewhat inconsistent depending on the situation. As such a formula may be applied to score the diverse selection.

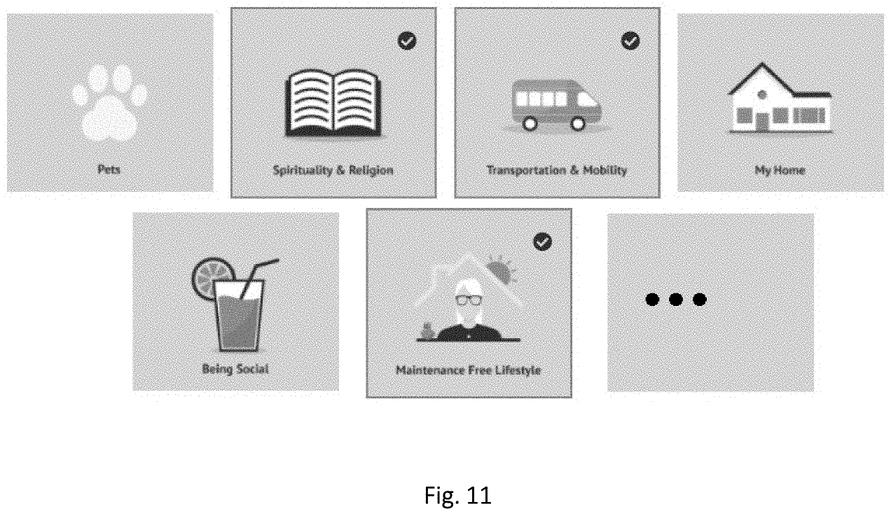

FIG. 11 is a block diagram illustrating a list of patient's requirements according to an embodiment of the disclosed subject matter. The user interface connected to the Care Matching System may display a list of items that important to the patient, i.e. patient requirements or strong preferences. These include pets, spirituality, transportation, my home, being social, maintenance free lifestyle and others. A patient may select one or more options. For example, in FIG. 11, the patient has selected spirituality, transportation, and maintenance fee lifestyle.

Although not shown in figures, the user interface connected to the Care Matching System may display a list of languages to the patient for selection. The patient may select only one language that is their primary language. This helps the Care Matching System in determining a match with a care facility that has care providers that speak the same language.

Additionally, as a next step, although not shown in figures, the user interface connected to the Care Matching System may display a budget tool for selection. Since budget may be an issue for a patient whose insurance does not cover the type of care needed, the budget tool allows the patient to select from a set of preset options or use a slider to customize and select a budget that suite them. The processor of the Care Matching System uses the budget selections, or data points form a slidable budget tool, to analyzes all the options and leverage the budget to get the patient the best possible match within the budget. If the cost is over, the Care Matching System provides the additional costs as well as options to get on a payment plan.

FIG. 12 is a block diagram illustrating a list of insurances that are to be selected by the patient according to an embodiment of the disclosed subject matter. The user interface connected to the Care Matching System may display a list of insurance options to determine the patient's insurance and all the insurances accepter by the care providers. Since figuring out insurance verification, insurance payout assessments, out-of-pocket costs, coverage, and other related costs is cumbersome and often complex for patients, the Care Matching System includes features under HIPAA guidelines to verify insurance, connect with required databases, both government, insurance, and private, over a secure network using security measures, the cumbersome work is eliminated and performed and completed by the Care Matching System with little work by the patient.

Options and choices presented in FIGS. 3-5 and 8-12 are also part of the decision matrix. The processor analyses all the choices and options selected, processes them through a decision tree to form a decision matrix. This matrix is weighted based on several factors including a weight of low, medium, and high for each option selected, any inconsistencies or conflicting selections.

The processor also makes decisions by navigating the course of the analysis by selecting one of the decision branches based on the options selected. The path varies based on each option selected. In the end, the result reflects the weighted analysis and options selected to determine an optimal care facility for the patient.

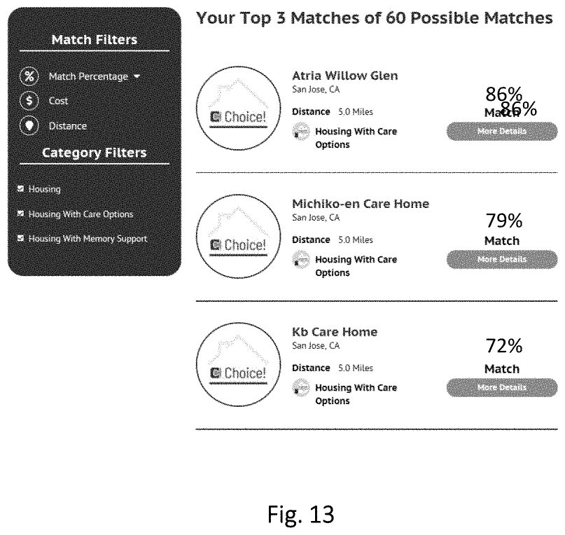

FIG. 13 depicts the results of the analysis performed by the Care Matching System according to an embodiment of the disclosed subject matter. The results are listed in a high to low matching percentage order with the highest percentage match on the top and the lowest percentage match on the bottom. Other formats and listing order are also contemplated. The user interface connected to the Care Matching System may also provide filtering options for the patient to sort and display the data on their device in a customizable format.

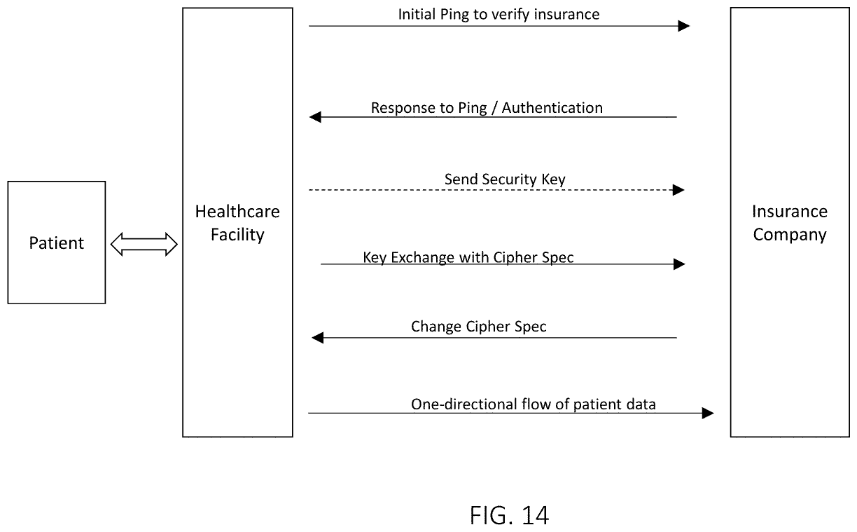

FIG. 14 depicts a method for patient's insurance verification according to an embodiment of the disclosed subject matter. The figure depicts a process for sending insurance patient data by using security and cipher according to one of the embodiments of the present invention. In this embodiment, an initial ping is sent to verify insurance. This ping may not contain confidential data and just a ping to get a response.

Then the receiving entity requesting information that will authorize and authenticate the receiver. In response the receiver sends the requested information which is then checked and authorizes and authenticates the receiver if matched. Once matched the sender separately sends a security key and also a key exchange of chippers occurs. The matching and deciphering of the ciphers and security key unlocks the one-directional data transfer from sender to receiver.

Exchange of security keys and ciphers between the patient, care giving entity, and the insurance company (or insurance carrier such as Medicare, Medicaid) is performed. Upon successful completion, patient insurance data is exchanged and the coverage and costs are exchanged. The method uses encryption, security keys, and ciphers and then exchanges information under HIPAA guidelines.

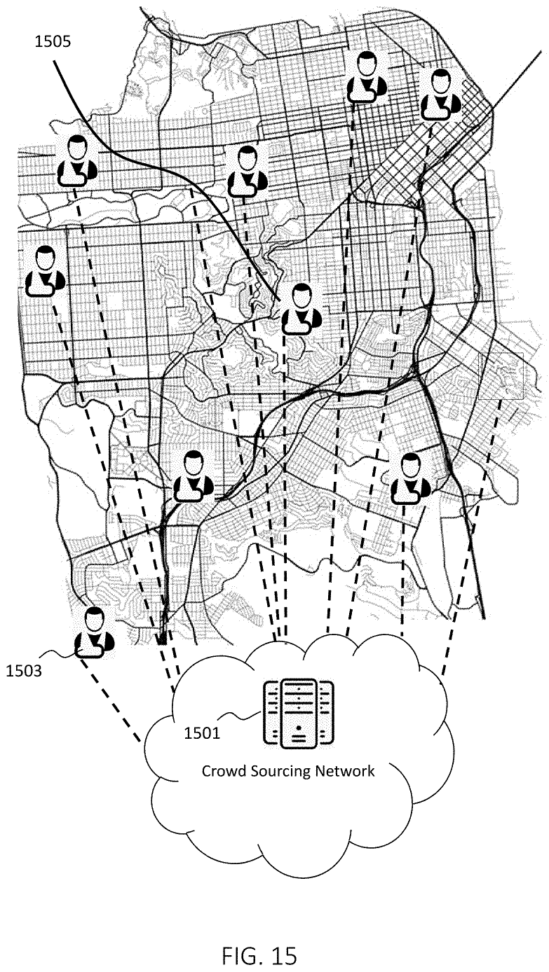

FIG. 15 depicts a crowd sourced network within a city according to an embodiment of the disclosed subject matter. The exemplary figure depicts the layout of the city of San Francisco. Other locations and geographies are contemplated. As shown in the figure, several patients, such as patient 1503, are connect to the crowd sourced server 1501. The patient may be spread across the city and sometime even farther.

The crowd sourced server 1501 collects data from each patient and their needs, preferences, and requirements, as well as the offerings of the care facilities. The crowd sourced server 1501 also collects match data, customer satisfaction data after admission, and rankings, complaints, and reviews. If the patient using the system is patient 1505 located in the central part of the city, the crowd sourced server 1501 collects the data with respect to distance from the server as well as local vicinity to curate crowd source data that is relevant based on location as well as other search criteria defined in Figs above. The crowd sourced server 1501 works with deep learning mechanisms of the Care Matching System to enhance the data for a specific patient and get a better match.

Comments from other patient, including details that are generally not posted by the care facility or review boards, such as specific day to day workings operations of their facility by room and care giver may be available—it depends on the type of data submitted by the patients. The data may provide the user of the system additional insight before selecting a care facility.

Although there are several advantages are stated in the details of this application above, the application is not so limited to these key advantages and include the details mentioned above as well as their obvious variations that are within the spirit and scope of the unique features mentioned.

1. A computer-implemented method for matching a patient to a caregiver or a care giving facility, the computer-implemented method comprising:
using an electronic device to enter patient care information, wherein the patient care information includes patient location, housing options, personal care options, insurance options, and additional option;
establishing a connection between the electronic device and a server of a Care Matching System, wherein said connection is over a network and said connection allows the user of the electronic device to communicate with the Care Matching System, wherein the server of the Care Matching System includes a processor and memory for storing data;
receiving the patient care information by server of the Medical Needs Matching and storing the patient care information in the memory;
the processor compartmentalizing the patient care information into a plurality of categories, wherein the categories include housing, personal care, insurance, additional option;
establishing a connection over a network between the server of the Care Matching System and a plurality of client computing devices connected to either a care giver or a care giving facility;
the processor performing a search query of care givers or care giving facilities within a predetermined distance from the patient location;
receiving a response to the query from the plurality of client computing devices connected to either a care giver or a care giving facility, wherein the response includes information relating to housing, personal care, and insurance;
the processor analyzing each care giver or care giving facility for its response to the housing information, wherein said analyzing includes determining a percentage match between the housing options entered using the electronic device and the housing related response provided by the care giver or care giving facility;
the processor assigning a housing score to each care giver or care giving facility that responded based on the percentage match determined;
the processor analyzing each care giver or care giving facility for its response to the personal care information, wherein said analyzing includes determining a percentage match between the personal care options entered using the electronic device and the personal care related response provided by the care giver or care giving facility;
the processor assigning a personal care score to each care giver or care giving facility that responded based on the percentage match determined;
the processor analyzing each care giver or care giving facility for its response to the insurance information, wherein said analyzing includes determining a percentage match between the insurance options entered using the electronic device and the insurance related response provided by the care giver or care giving facility;
the processor assigning an insurance score to each care giver or care giving facility that responded based on the percentage match determined;
the processor analyzing each care giver or care giving facility for its response to the additional option, wherein said analyzing includes determining a percentage match between the additional option entered using the electronic device and the additional option related response provided by the care giver or care giving facility;
the processor assigning an additional option score to each care giver or care giving facility that responded based on the percentage match determined;
the processor weighing the housing score, personal care score, insurance score, and additional option score to calculate a final score for each care giver or care giving facility;
the processor ranking each care giver or care giving facility and transferring the ranked results from the server of the Care Matching System to the display screen of the electronic device; and
the processor allowing the user of the electronic device to review the ranked care givers and care giving facilities and select one of the care givers or care giving facilities for onboarding the patient.

2. The computer-implemented method of claim 1, wherein the electronic device to enter patient care information is a mobile phone, wherein the mobile phone is connected to the Internet and is capable of downloading a mobile application, such mobile application being stored in the memory of a mobile device after being downloaded onto the mobile phone, such mobile application being downloaded from the server of the Care Matching System over a wireless network onto a mobile device, and wherein the mobile application providing access to the Care Matching System for uploading patient care information.

3. The computer-implemented method of claim 2, wherein the Care Matching System requiring the user of the mobile application to login using a unique username and password, wherein the Care Matching System requiring the user of the mobile application to upload basic information using the mobile application, the information includes location, housing information, personal care information, and insurance information.

4. The computer-implemented method of claim 3, wherein the user of the mobile application may enter additional information, wherein the additional information relates to patient's medical needs, patient's preferences, patient's requirements, patient's care options, patient's primary needs, patient's top concerns, patient's lifestyle interests, patient's ethnic and cultural requirements, patient's memory care, patient's nursing and rehabilitation care, patient's budget, patient's language preferences, and patient's care giver preference.

5. The computer-implemented method of claim 1, wherein the processor of the Care Matching System displays a wizard when the electronic device connects with the server through the Internet, wherein the wizard provides selectable options for the user of the electronic device, wherein one or more selections made by the user determine the match between the user's selections and the care giver or the care giving facility.

6. The computer-implemented method of claim 5, wherein the user of the electronic device may select one or more of the options presented by the wizard, wherein the options relates to patient's medical needs, patient's preferences, patient's requirements, patient's care options, patient's primary needs, patient's top concerns, patient's lifestyle interests, patient's ethnic and cultural requirements, patient's memory care, patient's nursing and rehabilitation care, patient's budget, patient's language preferences, and patient's care giver preference.

7. The computer-implemented method of claim 1, wherein the addition option includes memory care.

8. The computer-implemented method of claim 1, wherein the addition option relates cost for patient's care.

9. The computer-implemented method of claim 8, further comprising an electronic slidable budget tool, wherein the processor of the Care Matching System causes the electronic slidable budget tool to be presented on the display screen of the user's electronic device; wherein the user can slide the electronic budget tool from one location to another location to select a budget, wherein the processor uses the selected budget to find a care giver or a care giving facility that can provide their services within the selected budget.

10. The computer-implemented method of claim 1, wherein the addition option relates to one or more of the following categories: patient's medical needs, patient's preferences, patient's requirements, patient's care options, patient's primary needs, patient's top concerns, patient's lifestyle interests, patient's ethnic and cultural requirements, patient's memory care, patient's nursing and rehabilitation care, patient's budget, patient's language preferences, and patient's care giver preference.

11. The computer-implemented method of claim 10, wherein the patient's lifestyle interests include arts and culture, books and reading, crafts and being creative, culinary and foodie, family and grandchildren, fitness, friends and socializing, gardening and flowers, history, music, nature, pets, photography, puzzles and games, quiet time, science and technology, sports, sweets, and beach.

12. The computer-implemented method of claim 10, wherein the patient's top concerns include pets, spirituality and religion, transportation and mobility, my home, being social, and maintenance free lifestyle.

13. The computer-implemented method of claim 1, further comprising crowd sourcing engine, wherein the crowd sourcing engine is a feature of the Care Matching System, wherein the crowd sourcing engine performs the steps comprising:
receiving patient care information that is entered into the electronic device;
selecting one category of patient care information from the patient care information;
establishing a connection with a plurality of patients within a vicinity from the location of the electronic device;
querying the plurality of patients for information relating to the selected category;
receiving a response from a subset of the queried patients, wherein the response relates to the selected category; and
extracting category related information from the response and a score for the a care giver or a caregiving facility.

14. The computer-implemented method of claim 13, further deep learning engine, wherein the deep learning engine is a feature of the Care Matching System, wherein the deep learning engine performs the steps comprising:
establishing a connection with the crowd sourcing engine;
obtaining data from the crowd sourcing engine on each query by the plurality of patients and their responses by the car givers or the care giving facilities; and
refining the ranked care givers and care giving facilities by either upgrading their rank or downgrading their rank based on the search results of the query and responses.

14. The computer-implemented method of claim 1, further comprising:
adding the housing score;
adding the personal care score;
adding the insurance score;
adding the additional option score; and
determining a weighted overall score for each care giver or care giving facility.

15. The computer-implemented method of claim 1, further comprising a method for verifying insurance, wherein said method comprises:
establishing a connection over a secure network with an insurance carrier;
the processor of the Care Matching System sending an initial ping to the insurance carrier;
receiving a response to the ping and authenticating a secure connection;
the processor sending over the network a security key;
the processor over the network sending key exchange and cipher specification;
receiving a response from the insurance company with a change cipher specification signifying a secure connection;
the processor of the Care Matching System sending patient insurance information after the secure connection is established.

16. The computer-implemented method of claim 15, wherein the method for verifying insurance results in confirming that a specific care giver or care provider accepts the insurance of a specific patient.

17. The computer-implemented method of claim 1, wherein onboarding the patient includes:
the processor sending a medical document for completion to the electronic device;
the processor sending an administrative document for completion to the electronic device;
the processor sending a legal document for completion to the electronic device;
receiving an electronically signature for the medical, administrative, and legal documents; and
confirming completion of requirements to signal that the patient is ready for admission into a care giving facility.

18. The computer-implemented method of claim 18, further comprising providing an admission date to the user of the electronic device.

19. The computer-implemented method of claim 18, further comprising scheduling a tour date with the user of the electronic device.

20. The computer-implemented method of claim 1, wherein the weighing of the score is dependent upon each category being ranked low, medium, or high, wherein a low is weighted less than a high.
